(19) United States
(12) Patent Application Publication
(10) Pub. No.: US 2023/0380791 A1
(43) Pub. Date: Nov. 30, 2023

(54) METHOD FOR PLANNING A RECORDING OF AN X-RAY IMAGE OF A PATIENT BY AN X-RAY SYSTEM, AND OVERALL SYSTEM

(71) Applicant: Siemens Healthcare GmbH, Erlangen (DE)

(72) Inventor: Alois Regensburger, Poxdorf (DE)

(21) Appl. No.: 18/198,924

(22) Filed: May 18, 2023

(30) Foreign Application Priority Data
May 24, 2022 (DE) 10 2022 205 190.4

(51) Int. Cl. A61B 6/00; A61B 6/04

(52) U.S. Cl. CPC A61B 6/547; A61B 6/0407; A61B 6/467; A61B 6/4441

(57) ABSTRACT

A method for planning a recording of an X-ray image of a patient by an X-ray system. The X-ray system includes a recording system that may be adjusted into a large number of projection geometries with an X-ray source and an X-ray detector, using a mobile device comprising a display area. The method includes creating at least one first recording of a recording region of the patient by a capturing apparatus, for example an optical camera, providing a patient model of the patient anatomy of the patient, capturing the position and orientation of the manually positioned mobile device, creating a virtual X-ray image of at least one part of the patient using the patient model, at least the first recording and the position and/or orientation of the mobile device, and displaying the virtual X-ray image on the display area of the mobile device.

### CROSS REFERENCE TO RELATED APPLICATIONS

This application claims the benefit of DE 10 2022 205 190.4 filed on May 24, 2022, which is hereby incorporated by reference in its entirety.

### FIELD

Embodiments relate to a method for planning a recording of an X-ray image of a patient by an X-ray system.

### BACKGROUND

In medical 2D X-ray imaging, recording systems with a mounting which may be adjusted by motor, for example a C-arm for mounting an X-ray tube and an X-ray detector, are widely used. C-arms of this kind may be arranged for mapping the respective examination region in various projection geometries in relation to the examination object, for example in various projection directions, positions, orientations, and distances in relation to the examination object. Furthermore, recording systems also exist with X-ray source and X-ray detector that may be adjusted by motor independently of one another, for example the Siemens Multitom Rax.

Manually setting the projection geometry on the part of experienced medical-technical radiology assistants (MTRAs) or radiologists often does not yield an optimal result. In order to obtain an optimal projection geometry for the desired image when planning a two-dimensional projection image of the examination object, for example, test images with a low dose are often prepared from one or more projection directions, from which the optimal projection geometry and the optimal recording region are determined. In this context, however, additional radiation is used, to which the patient is exposed, which should generally be avoided. It is also known to use a patient model, in order to simulate a virtual X-ray recording using a piece of planning software after setting a projection geometry.

### BRIEF SUMMARY AND DESCRIPTION

The scope of the present disclosure is defined solely by the appended claims and is not affected to any degree by the statements within this summary. The present embodiments may obviate one or more of the drawbacks or limitations in the related art.

Embodiments provide a method that enables a rapid and intuitive planning of a recording geometry without the use of radiation and an X-ray system suitable for performing the method.

According to an embodiment, a method for planning a recording of an X-ray image of a patient by an X-ray system is provided.

The method according to an embodiment for planning a recording of an X-ray image of a patient by an X-ray system, wherein the X-ray system has a recording system that may be adjusted into a large number of projection geometries with an X-ray source and an X-ray detector, using a mobile device including a display area, includes the following steps: creating at least one first recording of a recording region of the patient by a capturing apparatus, for example an optical camera, providing a patient model of the patient anatomy of the patient, capturing the position and orientation of the manually positioned mobile device, creating a virtual X-ray image of at least one part of the patient using the patient model, at least the first recording and the position and/or orientation of the mobile device, and displaying the virtual X-ray image on the display area of the mobile device. By using the method, it is possible to find an optimal projection geometry for a desired recording of an X-ray image, in a manner that is simple and intuitive for an operator. In this context, it is possible to dispense with the use of X-ray radiation, meaning that it is possible to lower the radiation exposure of the patient, and possibly also of the operator, compared to the use of test recordings. Overall, in this manner the operator may try out a large number of projection geometries in a rapid and simple manner, without actually moving the recording system: the operator moves the mobile device manually and, by way of the method, anatomically correct virtual X-ray images are created automatically and displayed on the correspondingly positioned mobile device (or the display area thereof). If the operator moves the mobile device, then a new virtual X-ray image may be generated and displayed.

Generating or simulating a virtual X-ray image may be performed, for example, using simulation algorithms, for example Monte Carlo algorithms, or using machine learning algorithms or with computer graphics methods such as rasterization or ray tracing. In the representation, the virtual X-ray image may be modeled on a real X-ray image, or may also contain anatomical structures represented in color, may be a cinematic rendering representation, or may also show anatomical structures that cannot be or may only be very weakly contrasted in the X-ray image. In the computer graphics methods, for example, only the skeleton of the patient and the soft tissue mass may be represented in a simplified model.

According to an embodiment, the virtual X-ray image is determined in such a manner that the position and/or orientation of the mobile device at least partially represent the position and/or the orientation of the X-ray detector. Thus, for example, the virtual X-ray image may be determined in such a manner that the position and orientation, or just the position, of the mobile device corresponds to the X-ray detector, if it were arranged at the same position. For example, the display area of the mobile device may also at least partially represent the position and/or orientation of the sensor area of the X-ray detector. For example, the virtual X-ray image may also be determined in such a manner that the center of the display area of the mobile device corresponds to the center of the sensor area of the X-ray detector, if it were arranged at the same position, with a reduced image area or scaled image size relative to one another. Also, to determine the virtual X-ray image, the distance between patient and X-ray detector may correspond to the distance between mobile device and patient, or this may be variable or specified at a fixed value in advance (for example, the SID (=source image distance, distance between X-ray source and X-ray detector) may also be fixed). In this manner, the operator may simulate a position and/or orientation of the X-ray detector by the method and may see how a possible X-ray image changes at different X-ray detector positions.

According to an embodiment, the virtual X-ray image is alternatively determined in such a manner that the position and/or orientation of the mobile device at least partially represent the position and/or the orientation of the X-ray source. Here, the center of the display area may correspond to the focal point, for example. The distance between mobile device and patient may correspond to the distance between X-ray device and patient, or this may be variable or specified at a fixed value in advance (for example, the SID may also be fixed). In this manner, the operator may simulate the influence of the position and/or the orientation of the X-ray source on the X-ray image.

According to an embodiment, at least one parameter is determined for the X-ray system for recording an X-ray image, which X-ray image at least partially corresponds to the virtual X-ray image. For example, a positioning of the recording system and/or a patient couch assigned to the X-ray system may be determined for recording the X-ray image at least partially corresponding to the virtual X-ray image. For this purpose, for example, it is possible to use the specification for the position and/or orientation of the X-ray detector or the X-ray source used further above and to determine a positioning for the recording system that meets the specification. It is also or additionally possible for parameters such as dose, X-ray voltage or collimation to be determined on the basis of the virtual X-ray image. For these parameters, it is also possible to take into consideration other criteria, for example as low a radiation exposure as possible with simultaneously good image quality, or similar. A determination of the parameters may be determined automatically or based on an input.

According to an embodiment, the patient model of the, for example, internal patient anatomy, i.e. bones, organs, vessels etc. for example, is retrieved from a memory or a database, or is ascertained from a previously recorded volume image of the patient. The patient model may be a rather general model, an adapted model that has been created using some details regarding the patient (sex, height, weight, etc.), or an exact model that has been defined more precisely on the basis of previous X-ray images (2D or 3D). For example, it may involve a digital twin model. The patient model therefore also influences the precision of the virtual X-ray image.

According to an embodiment, the X-ray system may subsequently be actuated to record an X-ray image with the at least one parameter. In order to trigger this, it is possible for an additional input (for example by an operator) to be received, for example. The recording system and/or the patient couch are actuated, adjusted into the corresponding positions, and all necessary or previously determined parameters are set. Subsequently, an application of X-ray radiation is triggered. A collision monitoring may also be used in advance.

According to an embodiment, the mobile device is formed by a smartphone, smart device, touchpad, or tablet. Mobile devices of this kind are particularly suitable for use for the method, as they are light, readily available, and well equipped from a technical perspective, for example having a touch display, a camera, and a tracking system. The virtual X-ray image may be displayed on the display area, i.e., the touch display. Mobile devices of this kind, for example provided with an app, may also be used to operate the X-ray system. It is possible, for example, for settings such as collimation, zoom of the X-ray image, dose, and the like to be set via the app, that for example may also display the virtual (for example simulated) X-ray image.

According to an embodiment, the position and/or the orientation of the mobile device are determined using radio locating and/or visual locating by markings and/or via a tracking system. Thus, for example, locating of a mobile device may be performed as in the aforementioned examples (smartphone, smart device, touchpad, or tablet) via a radio network (WLAN, 5G, 4G, GPS), as the necessary prerequisites for this are already available in commercially available mobile devices. It is likewise possible to use other simple and precise possibilities for locating or tracking, such as via visual markings in the room and/or on the X-ray system or via a tracking attachment on the mobile device (for example USB-C dongle), for example. An orientation of the mobile device may be determined via proximity sensors integrated in the mobile device, for example.

According to an embodiment, the steps of capturing the position and orientation of the manually positioned mobile device, creating, and displaying a virtual X-ray image are repeated until a control signal is received. The control signal may involve an input by an operator, for example, or an automatic control signal, if an optimal virtual X-ray image is detected for an intended X-ray recording. By way of the control signal, for example, it is possible to receive a trigger, by way of which a determination of the parameter(s) is initiated.

Embodiments also include an overall system for performing the method, including an X-ray system with a recording system that may be adjusted into a large number of projection geometries with an X-ray source and an X-ray detector, a mobile device with a display area, a capturing apparatus, for example an optical camera, for capturing at least one first recording of a recording region of a patient, a provision unit for providing a patient model of the patient anatomy, a position determining system for determining the position and orientation of the manually positioned mobile device, and a calculating unit for creating a virtual X-ray image of at least one part of the recording region of the patient using the patient model, at least the first recording and the position and/or orientation of the mobile device, wherein the display area is configured to display the virtual X-ray image.

According to an embodiment, the overall system includes a determining unit for determining at least one parameter for the X-ray system for recording an X-ray image, which X-ray image at least partially corresponds to the virtual X-ray image. For example, the determining unit is configured to determine a positioning of the recording system for recording the X-ray image. Advantageously, the overall system has an adjustable patient couch. This may likewise be adjusted in order to obtain an optimal projection geometry relative to the patient.

The overall system also includes a control unit, that is configured to actuate a recording of an X-ray image with the at least one parameter. Expediently, the mobile device is formed by a smartphone, smart device, touchpad, or tablet. According to an embodiment, the recording system is formed by an adjustable C-arm.

### BRIEF DESCRIPTION OF THE FIGURES

FIG. 1 depicts a sequence of steps of the method according to an embodiment.

FIG. 2 depicts a further sequence of steps of the method according to an embodiment.

FIG. 3 depicts a view of an overall system for performing the method according to an embodiment.

FIG. 4 depicts a view of the recording system of the overall system and the mobile device during the position determination of the mobile device according to an embodiment.

FIG. 5 depicts a top view of the mobile device, showing the virtual X-ray image according to an embodiment.

FIG. 6 depicts a view of the recording system of the overall system with X-ray detector adjusted in accordance with the position of the mobile device in FIG. 4 according to an embodiment.

FIG. 7 depicts a further sequence of steps of the expanded method according to an embodiment.

### DETAILED DESCRIPTION

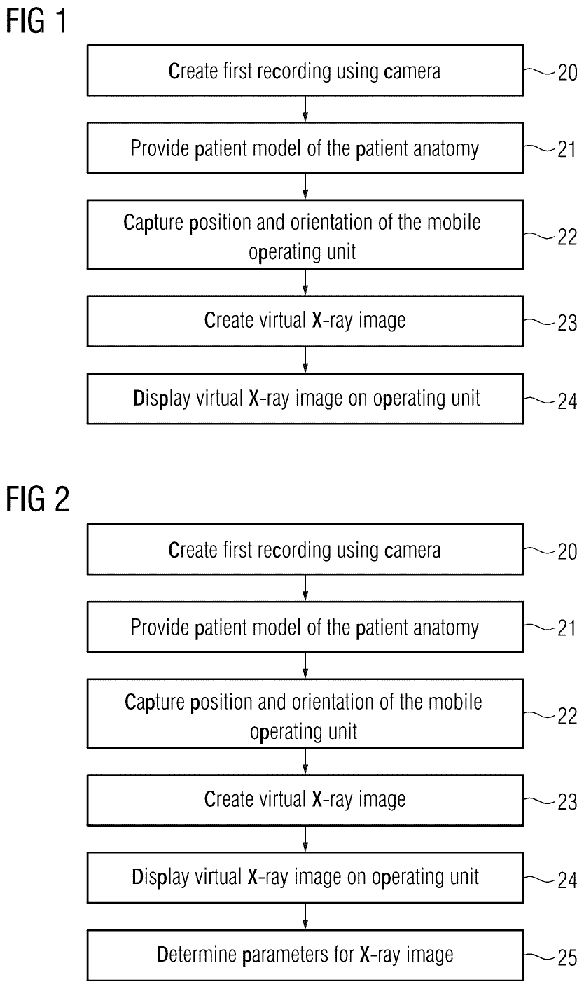

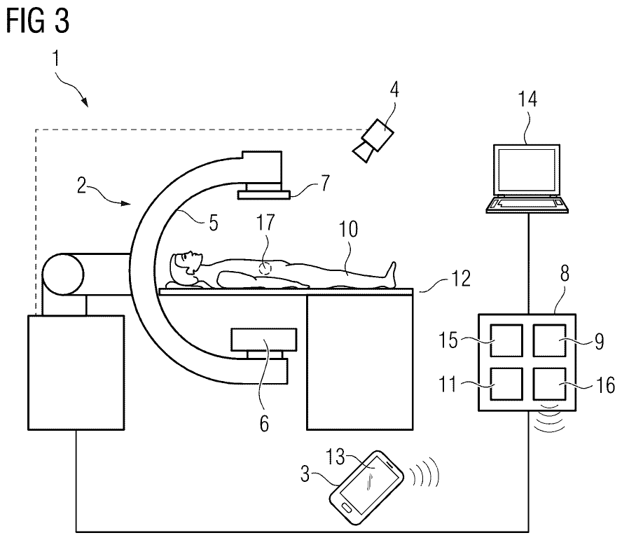

By way of the method, it is possible for an operator, without test recordings and therefore without the emission of X-ray radiation, in a simple and intuitive manner and with the aid of a mobile device, to gain an accurate idea of how a certain projection geometry affects the representation of the examination object 17 (for example an organ or examination region of a patient) on the X-ray image, and a particularly suitable projection geometry may be selected as a result. In this manner, inter alia, the radiation exposure of the patient is reduced, X-ray images may be recorded in a more rapid and exact manner, and time and money are saved due to the lower number of incorrect recordings. FIGS. 1 and 2 and 7 show three possible sequences of the method; FIG. 3 depicts an overall system 1 that may be used to perform the method with an X-ray system configured as a C-arm X-ray device 2 and a mobile device 3. The C-arm X-ray device 2 includes a C-arm 5, that may be adjusted in multiple spatial directions and on which an X-ray source 6 and an X-ray detector 7 are mounted. The mobile device 3 may involve a smartphone, smart device, touchpad, or tablet, for example, that has a display area 13 (for example touchscreen).

In a first step 20, at least one first recording of a recording region of the patient 10 is created by a capturing apparatus. This is used to capture the current positioning of the patient. To this end, an optical camera 4 may be used, for example a camera that is installed in a fixed manner in the examination room (and for example also registered with the X-ray device) or arranged on the X-ray system 2; a camera of the mobile device 3 may also be used. By the optical camera, it is also possible for more than one, i.e., for example two or more recordings to be captured. Alternatively, it is also possible to use other capturing apparatuses, for example a radar system, that captures the current position and/or orientation of the recording region of the patient. The recording may also be performed spaced apart from the rest of the method in terms of time, provided that the patient does not change his positioning in the meantime.

In a second step 21 of the method, a patient model of the patient anatomy of the patient is provided, for example by a provision unit 15. The patient model may, for example, be retrieved from a memory or a database or ascertained/generated from a previously recorded volume image of the patient. The patient model may involve a general statistical patient model, that has already been modified with information and data of the patient (height, weight, sex etc.), or a patient model that has been exactly adapted to the patient. In this context, the patient model may involve a digital twin model of the patient, for example. The patient model involves a three-dimensional patient model of at least one section of the internal anatomy of the patient, that contains the planned examination object 17. The patient model therefore contains information regarding the position of bones, organs, vessels and/or tissue of the patient, for example. Alternatively, the patient model may contain only information regarding the location of bones and the skin surface and may assume all the soft tissue outside of the bones to be a homogenous substance.

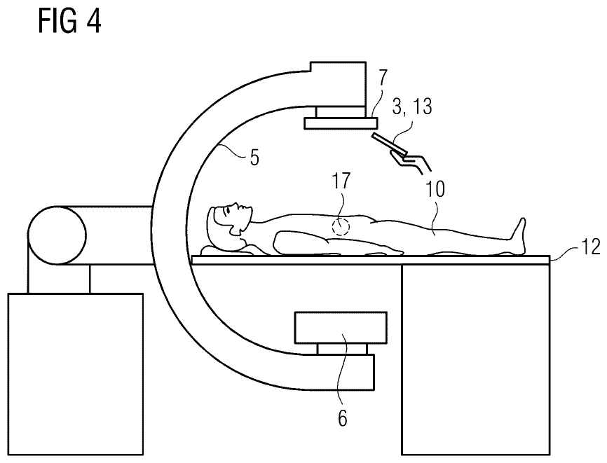

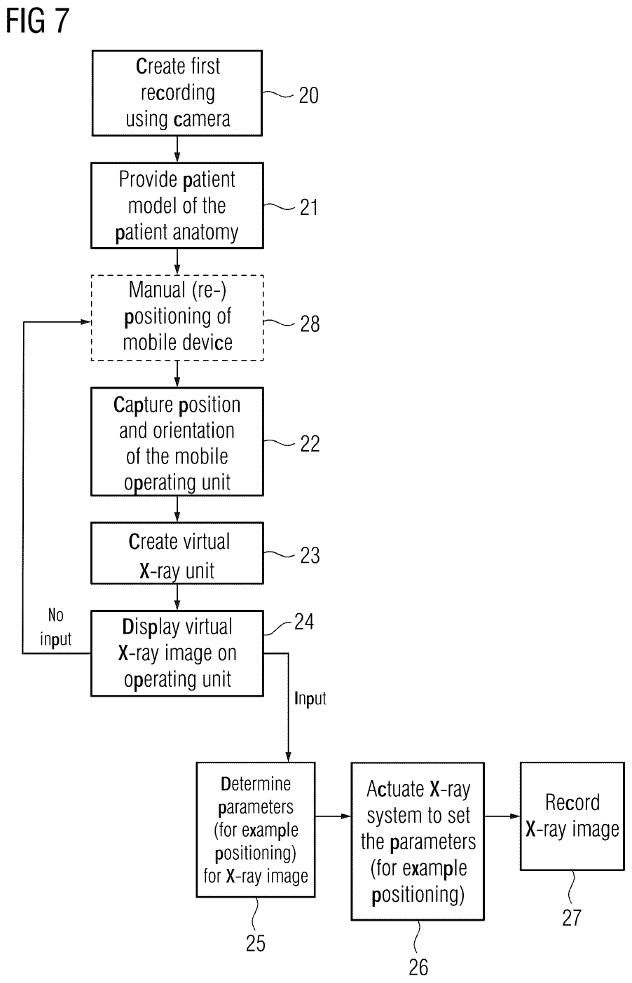

In a third step 22, the position and the orientation of the mobile device previously manually positioned is captured, a position determining system 16 being provided for this purpose, for example. The manual positioning may also be performed in an intermediate step 28—see FIG. 7—for example by an operator manually holding the mobile device in the desired position and orientation (see also FIG. 4 for example). The position capturing may be determined using radio locating and/or visual locating by markings and/or via a tracking system. Thus, for example, a locating of the mobile device (for example smartphone, smart device, touchpad, or tablet) may be performed via a radio network, since the necessary prerequisites for this are available in commercially available mobile devices. It is likewise possible to use other simple and precise possibilities for locating or tracking, such as via visual markings in the room and/or on the X-ray system or via a tracking attachment on the mobile device (for example USB-C dongle), for example. The operator may hold the mobile device, for example with their hand or an aid (for example telescopic rod), and position it accordingly. In this context, the operator holds the mobile device, for example, similarly to how the X-ray detector or the X-ray source are to be positioned later, for example in terms of the position, orientation, projection direction and/or the distance.

In a fourth step 23, a virtual X-ray image of at least one part of the recording region of the patient (for example of the region that is relevant or of interest) is created from the information provided in the first three steps, i.e., the at least one camera image, the patient model of the internal anatomy of the patient and the position and/or orientation of the mobile device. The virtual X-ray image may be calculated or simulated, for example, by a calculating unit 9 using software algorithms, for example also using machine learning software. The virtual X-ray is created in such a manner that the projection geometry of the virtual X-ray image (relating to the X-ray system used) depends upon the captured position and orientation of the mobile device 3. Depending upon a previously chosen setting or an input by an operator for example, there are various possibilities for this. Thus, for example, the virtual X-ray image may be determined in such a manner that the position and/or orientation of the mobile device 3 at least partially corresponds to the position and/or the orientation of the X-ray detector 7 of the X-ray system.

The virtual X-ray image may be simulated in such a manner that the display area 13 of the mobile device 3 or the center thereof corresponds to the sensor area of the X-ray detector 7 or the center of the sensor area. For example, the X-ray detector 7 may be larger than the virtual X-ray image shown; the virtual X-ray image may be reduced in size, true to scale, in relation to the display area 13. The orientation of the display area 13 of the mobile device 3 may likewise correspond to the orientation of the X-ray detector 7 in the virtual X-ray image. In this case, the location of the X-ray source 6 may be assumed to be “opposite” the X-ray detector, i.e. on the other side of the patient, starting from the central axis of the X-ray detector 7. The central projection beam of the X-ray source 6 of the virtual X-ray image should strike the sensor area of the X-ray detector 7 in a perpendicular manner. A collimation of the virtual X-ray image may be chosen, for example, such that the X-ray detector 7 (or a previously selected subregion) is illuminated with X-ray radiation.

The distance of the X-ray source from the X-ray detector (SID) of the virtual X-ray image may be specified in a fixed manner, for example analogously to the X-ray system. If the SID is variable in the X-ray system, then initially a standard value may be chosen on an automatic basis. By way of user input, the SID may be selected with increased or reduced size.

Alternatively, the virtual X-ray image may be determined in such a manner that the position and/or orientation of the mobile device 3 at least partially corresponds to the position and/or the orientation of the X-ray source 6 of the X-ray system. The position of the X-ray detector 7 of the virtual X-ray image is then specified analogously to the description above. For example, the center of the display area 13 of the mobile device on the virtual X-ray image may correspond to the focus of the X-ray source 6.

In a fifth step 24, the virtual X-ray image generated in this manner is subsequently displayed on the display area 13 of the positioned mobile device 3—see also FIG. 5, for example. The virtual X-ray image may be visually identical to an actual X-ray image or may be represented such that it may be clearly identified as a substitute, for example as schematic structures, such as bones and organs for example, overlaid on the first recording (for example camera image), or as an augmented reality representation on the first recording.

Thus, on the basis of the virtual X-ray image displayed on the mobile device 3, the operator may see how a corresponding X-ray recording would appear. The operator may then decide, for example, whether or not they would like to have an X-ray image of this kind and may initiate corresponding actions. The virtual X-ray image may also additionally be shown on a further display unit 14 (for example monitor).

If the method is repeated, then the first recording (first step 20) and the provision of the patient model (second step 21) do not have to be repeated, provided that the same patient remains in the same position.

In a further embodiment of the method—see FIG. 2—in a sixth step 25 subsequently a parameter for the X-ray system is determined for recording an X-ray image at least partially corresponding to the virtual X-ray image, for example by a determining unit 11. The parameters may involve a positioning of the X-ray source 6 and the X-ray detector 7 (or the recording system, for example C-arm 5), for example, and/or a positioning of a patient couch 12, on which the patient 10 is positioned. This may also involve an X-ray voltage and/or X-ray dose of the X-ray source or a collimation by a collimator.

FIG. 7 depicts a method, in which initially all steps up to the fifth step 24, the displaying of the virtual X-ray image, are performed. Other than the manual positioning of the mobile device 3, the steps take place automatically. If no (specific) input takes place, then the position/orientation determination of the mobile device (third step 22), the calculation of the virtual X-ray image (fourth step 23) and the display of the virtual X-ray image (fifth step 24) are repeated either continuously or at (regular) intervals. The operator may thus try out various positions/orientations of the mobile device and thus various projection geometries and have the corresponding virtual X-ray image displayed. If the virtual X-ray image displayed appears to the operator to be appropriate for an actual recording, then the operator may perform an input and thus trigger the determination of the parameter(s) (sixth step 25), for example by way of a control signal. A triggering may also take place automatically, for example if the current virtual X-ray image is analyzed automatically and is assessed to be appropriate or optimal for an intended X-ray recording.

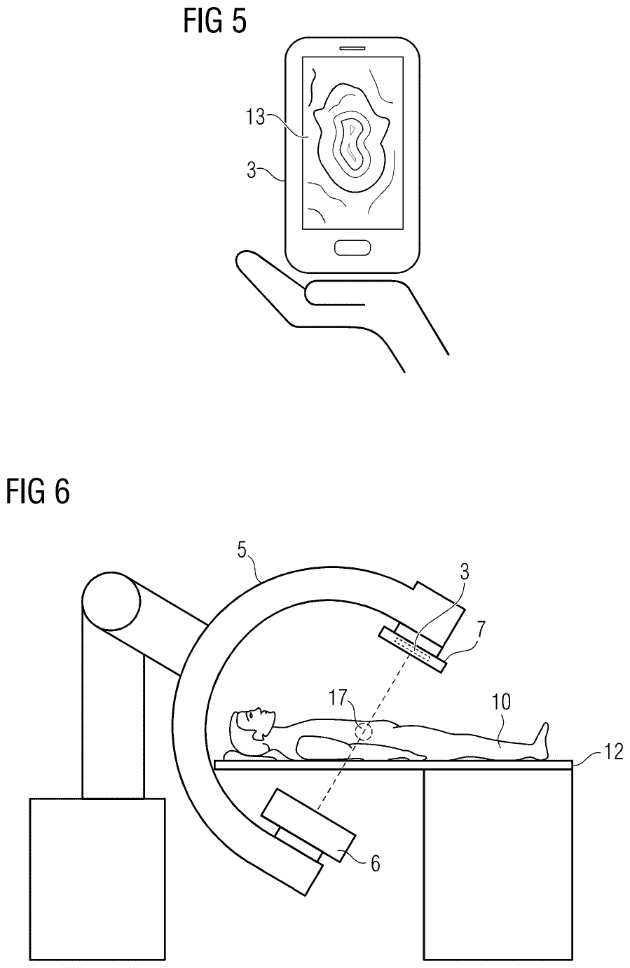

Subsequently, in a seventh step, the X-ray system may actuate the setting of the determined parameters, for example by the system control unit 8, for example adopting the position of the recording system (for example the corresponding projection geometry), the patient couch 12 and/or setting X-ray voltage, dose, and collimation. FIG. 6 depicts how the X-ray detector 7 is adjusted into the same position and orientation previously occupied by the mobile device—see FIG. 4. In an eighth step 27, an X-ray image is then recorded with the set parameters.

In addition to a C-arm X-ray device, the X-ray system may also be formed by various other X-ray devices, for example for fluoroscopy, in which the X-ray source and the X-ray detector are configured such that they may be adjusted into various projection geometries. This includes X-ray devices with X-ray source and X-ray detector that are mounted together or separately, wherein both may be adjusted either together or separately in multiple spatial directions.

The mobile device is formed by a smartphone, smart device, touchpad, or tablet, for example. Mobile devices of this kind are particularly suitable for use for the method, as they are light, readily available, and well equipped from a technical perspective, for example having a touch display, a camera, and a tracking system. The virtual X-ray image may be displayed on the display area, i.e., the touch display. Mobile devices of this kind, for example provided with an app, may also be used to operate the X-ray system.

The at least one first recording (for example by the camera) and the capturing of position and/or orientation of the mobile device may furthermore also be used for collision avoidance, for example by impermissible positions for the X-ray source and the X-ray detector being detected and automatically excluded.

For a particularly intuitive, time-saving and low-radiation X-ray recording, a method for planning a recording of an X-ray image of a patient by an X-ray system, wherein the X-ray system has a recording system that may be adjusted into a large number of projection geometries with an X-ray source and an X-ray detector, using a mobile device including a display area, is provided with the following steps: creating at least one first recording of a recording region of the patient by a capturing apparatus, for example an optical camera, providing a patient model of the patient anatomy of the patient, capturing the position and orientation of the manually positioned mobile device, creating a virtual X-ray image of at least one part of the patient using the patient model, at least the first recording and the position and/or orientation of the mobile device, and displaying the virtual X-ray image on the display area of the mobile device.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for planning a recording of an X-ray image of a patient by an X-ray system, wherein the X-ray system includes a recording system that may be adjusted into a large number of projection geometries with an X-ray source and an X-ray detector, using a mobile device comprising a display area, the method comprising:
creating at least one first recording of a recording region of the patient by a capturing apparatus;
providing a patient model of a patient anatomy of the patient;
capturing a position and an orientation of the mobile device that is manually positioned;
creating a virtual X-ray image of at least one part of the patient using the patient model, the at least one first recording, and the position, the orientation, or the position and the orientation of the mobile device; and
displaying the virtual X-ray image on the display area of the mobile device.

2. The method of claim 1, wherein the virtual X-ray image is determined such a manner that the position, the orientation, or the position and the orientation of the mobile device at least partially represent a position, an orientation, or the position and the orientation of the X-ray detector.

3. The method of claim 1, wherein the virtual X-ray image is determined in such a manner that the position, the orientation, or the position and the orientation of the mobile device at least partially represent a position, an orientation, or the position and the orientation of the X-ray source.

4. The method of claim 1, wherein at least one parameter is determined for the X-ray system for recording an X-ray image, which X-ray image at least partially corresponds to the virtual X-ray image.

5. The method of claim 4, wherein a positioning of the recording system, a positioning of a patient couch assigned to the X-ray system, or a positioning of the recording system and a positioning of a patient couch assigned to the X-ray system may be determined for recording the X-ray image at least partially corresponding to the virtual X-ray image.

6. The method of claim 1, wherein the patient model of the patient anatomy is retrieved from a memory or a database or ascertained from a previously recorded volume image of the patient.

7. The method of claim 4, wherein the X-ray system is subsequently actuated to record an X-ray image with the at least one parameter.

8. The method of claim 1, wherein the position, the orientation, or the position and the orientation of the mobile device are determined using radio locating, visual locating, or radio locating and visual locating by markings, via a tracking system, or by markings and via the tracking system.

9. The method of claim 1, wherein the mobile device is a smartphone, smart device, touchpad, or tablet.

10. The method of claim 1, wherein the display area of the mobile device at least partially represents a position, an orientation, or the position and the orientation of a sensor area of the X-ray detector.

11. The method of claim 1, wherein the steps of capturing the position and orientation of the manually positioned mobile device, creating, and displaying a virtual X-ray image are repeated until a control signal is received.

12. A system comprising:
an X-ray system with a recording system which may be adjusted into a large number of projection geometries with an X-ray source and an X-ray detector;
a mobile device with a display area;
a capturing apparatus, for example an optical camera, for capturing at least one first recording of a recording region of a patient;
a provision unit for providing a patient model of a patient anatomy;
a position determining system for determining the position and orientation of the manually positioned mobile device; and
a calculating unit for determining a virtual X-ray image of at least one part of the recording region of the patient using the patient model, at least the first recording and the position, the orientation, or the position and the orientation of the mobile device;
wherein the display area of the mobile device is configured to display the virtual X-ray image.

13. The system of claim 12, wherein the calculating unit is configured to determine the virtual X-ray image in such a manner that the position, the orientation, or the position and the orientation of the mobile device at least partially represent a position, an orientation, or the position and the orientation of the X-ray detector.

14. The system of claim 12, wherein the calculating unit is configured to determine the virtual X-ray image in such a manner that the position, the orientation, or the position and the orientation of the mobile device at least partially represent a position, an orientation, or the position and the orientation of the X-ray source.

15. The system of claim 12, further comprising:
a determining unit configured for determining at least one parameter for the X-ray system for recording an X-ray image that at least partially corresponds to the virtual X-ray image.

16. The system claim 15, further comprising:
a patient couch.

17. The system of claim 15, wherein the determining unit is configured to determine a positioning of the recording system for recording the X-ray image.

18. The system of claim 15, further comprising:
a control unit configured to actuate a recording of an X-ray image with the at least one parameter.

19. The system of claim 12, wherein the mobile device is a smartphone, smart device, touchpad, or tablet.

20. The system of claim 12, wherein the recording system comprises an adjustable C-arm.
